(19) United States
(12) Patent Application Publication
(10) Pub. No.: US 2024/0041626 A1
(43) Pub. Date: Feb. 8, 2024

(54) DELIVERY DEVICE AND ENDOSCOPE SYSTEM

(71) Applicant: OLYMPUS MEDICAL SYSTEMS CORP., Tokyo (JP)

(72) Inventor: Yoichi SAKURADA, Higashimurayama-shi (JP)

(21) Appl. No.: 18/490,342

(22) Filed: Oct. 19, 2023

Related U.S. Application Data
(63) Continuation of application No. PCT/JP21/20470, filed on May 28, 2021.

(51) Int. Cl. A61F 2/966; A61F 2/95; A61B 1/00

(52) U.S. Cl. CPC A61F 2/966; A61F 2/9517; A61F 2/9522; A61B 1/00133; A61F 2002/9665

(57) ABSTRACT

The delivery device includes: a first outer tube member; an inner tube member that is inserted into the first outer tube member; a second outer tube member that is positioned at a proximal side with respect to the first outer tube member and is connected to the inner tube member; a pulling member that is inserted into the second outer tube member and is connected to the first outer tube member; a first joint member in which a first hole for fixing the pulling member therein and a second hole configured to allow the inner tube member to advance and retract are formed; and a fixing member that fixes the first joint member to the first outer tube member.

Exemplary embodiments relates to a delivery device and an endoscope system. This application is a continuation application based on International Patent Application No. PCT/JP2021/020470 filed on May 28, 2021 and the contents of the PCT international application is incorporated herein by reference.

### BACKGROUND

A typical example of a delivery device is a stent delivery device used for indwelling a stent in the body to dilate a stenosis or obstruction (hereinafter referred to as “stenosis or the like”) that occurs in the digestive tract or the like. The stent delivery device is inserted into a treatment instrument channel of an endoscope to deliver the stent to the stenosis or the like. After that, the stent delivery device indwells the stent stored within the device to the stenosis or the like.

In a stent delivery device described in Japanese Unexamined Patent Application, First Publication No. 2006-271565, a distal side tube is inserted into a tubular member, and the distal side tube is provided slidably with respect to the tubular member. A stent is stored between the tubular member and the distal side tube. The tubular member is moved by pulling a pulling member fixed to the tubular member and the stored stent is placed in the stenosis or the like.

### SUMMARY

According to a first aspect of the present disclosure, there is provided a delivery device including: a first outer tube member; an inner tube member that is inserted into the first outer tube member; a second outer tube member that is positioned at a proximal side with respect to the first outer tube member and is connected to the inner tube member; a pulling member that is inserted into the second outer tube member and is connected to the first outer tube member; a first joint member in which a first hole for fixing the pulling member therein and a second hole configured to allow the inner tube member to advance and retract are formed; and a fixing member that fixes the first joint member to the first outer tube member.

According to a second aspect of the present disclosure, there is provided an endoscope system including: a delivery device including a first outer tube member, an inner tube member that is inserted into the first outer tube member, a second outer tube member that is positioned at a proximal side with respect to the first outer tube member and is connected to the inner tube member, a pulling member that is inserted into the second outer tube member and is connected to the first outer tube member, a first joint member that has a first hole for fixing the pulling member therein and a second hole configured to allow the inner tube member to be inserted thereinto and to allow the inner tube member to advance and retract, and a fixing member that fixes the first joint member to the first outer tube member; and an endoscope having a treatment instrument channel configured to allow the delivery device to advance and retract.

### BRIEF DESCRIPTION OF THE DRAWINGS

FIG. 1 is a view showing an endoscope system.

FIG. 2 is a view showing the overall configuration of a delivery device of the endoscope system.

FIG. 3 is a view showing members constituting the delivery device.

FIG. 4 is a view showing members constituting the delivery device shown in FIG. 3 with a partially broken cross-sectional view.

FIG. 5 is a cross-sectional view showing a first joint portion of the delivery device.

FIG. 6 is a cross-sectional view along line C6-C6 shown in FIG. 5.

FIG. 7 is a cross-sectional view along line C7-C7 shown in FIG. 5.

FIG. 8 is a cross-sectional view showing a second joint portion of the delivery device.

FIG. 9 is a cross-sectional view along line C9-C9 shown in FIG. 8.

FIG. 10 is a cross-sectional view along line C10-C10 shown in FIG. 8.

FIG. 11 is a partial cross-sectional view of the delivery device.

FIG. 12 is a partial cross-sectional view showing a state in which a stent is released from the delivery device.

### DETAILED DESCRIPTION OF THE INVENTION

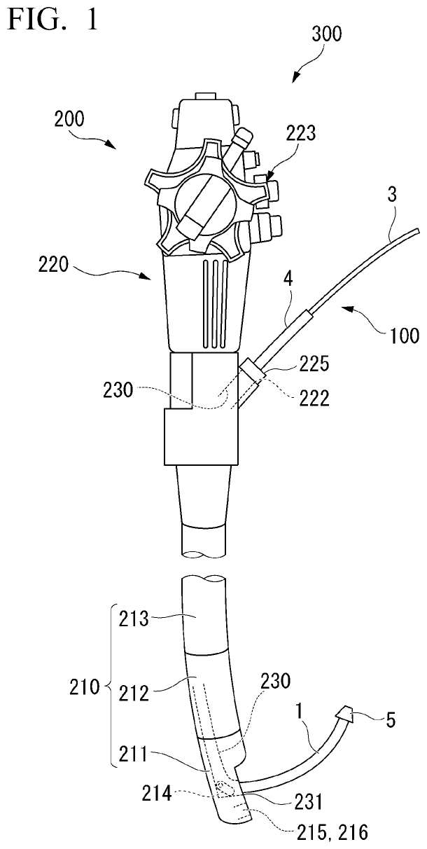

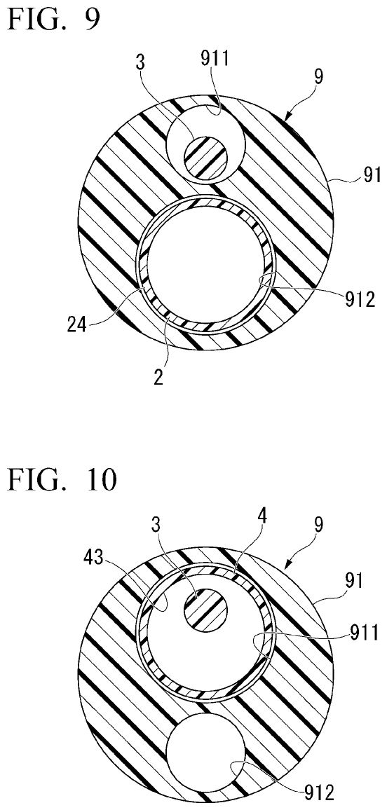

An embodiment of the present disclosure will be described with reference to FIGS. 1 to 12.

### [Endoscope System 300]

An endoscope system 300 will be described with reference to FIG. 1. The endoscope system 300 includes an endoscope 200 and a delivery device 100. The delivery device 100 is inserted into a channel of the endoscope 200.

### [Endoscope 200]

The endoscope 200 will be described with reference to FIG. 1.

The endoscope 200 is a known side-viewing flexible endoscope and includes a long insertion section 210, an operation section 220, and a treatment instrument channel 230. The operation section 220 is provided at a proximal end portion of the insertion section 210. In the following description, a side of the operation section 220 of the endoscope 200 is referred to as a proximal side. A side opposite to the operation section 220 in a longitudinal axis direction of the insertion section 210 is referred to as a distal side of the endoscope 200. A treatment instrument such as the delivery device 100 is inserted into the treatment instrument channel 230. The endoscope 200 may be a direct viewing flexible endoscope.

The insertion section 210 has a distal end rigid portion 211, a bending portion 212, and a flexible tube portion 213. The distal end rigid portion 211 is provided at a distal end portion of the insertion section 210. The bending portion 212 is attached to a proximal side of the distal end rigid portion 211 and is configured to be bendable. The flexible tube portion 213 is attached to the proximal side of the bending portion 212.

An imaging unit 216 is provided on a side surface of the distal end rigid portion 211 and exposed to the outside. The imaging unit 216 has a light guide 215 and a CCD.

The distal end rigid portion 211 is provided with an elevator 214. A proximal end portion of the elevator 214 is rotatably supported by the distal end rigid portion 211. An elevator operation wire (not shown) is fixed to a distal end portion of the elevator 214. The elevator operation wire (not shown) extends toward the proximal side through the insertion section 210.

The bending portion 212 is configured to be bendable in an up-down direction and a left-right direction. In the bending portion 212, a distal end of the operation wire is fixed to the distal side of the bending portion 212. The bending portion 212 is bent in a direction intersecting with an axis of the insertion section 210 in a state that the insertion section 210 extends straight. The operation wire extends through the insertion section 210 to the operation section 220. The up-down direction is an up-down direction of the field of view of the endoscope among orthogonal directions of the field of view. The left-right direction is a left-right direction of the field of view of the endoscope among orthogonal directions of the field of view. A bending direction of the bending portion 212 is not limited to the up-down direction and the left-right direction and may also be a direction intersecting with the axis of the insertion section 210.

A distal end portion 231 of the treatment instrument channel 230 opens on the side surface of the distal end rigid portion 211. A proximal end portion of the treatment instrument channel 230 extends to the operation section 220.

A proximal end portion of the operation section 220 is provided with a knob 223 for operating the operation wire and a switch 224 for operating the imaging unit 216, and the like. A user can bend the bending portion 212 in a desired direction by operating the knob 223.

A forceps port 222 that communicates with the treatment instrument channel 230 is provided on the distal side with respect to the operation section 220. The user can insert a treatment instrument for an endoscope such as the delivery device 100 through the forceps port 222. A forceps plug 225 is attached to the forceps port 222 in order to prevent leakage of a bodily fluid.

### [Delivery Device 100]

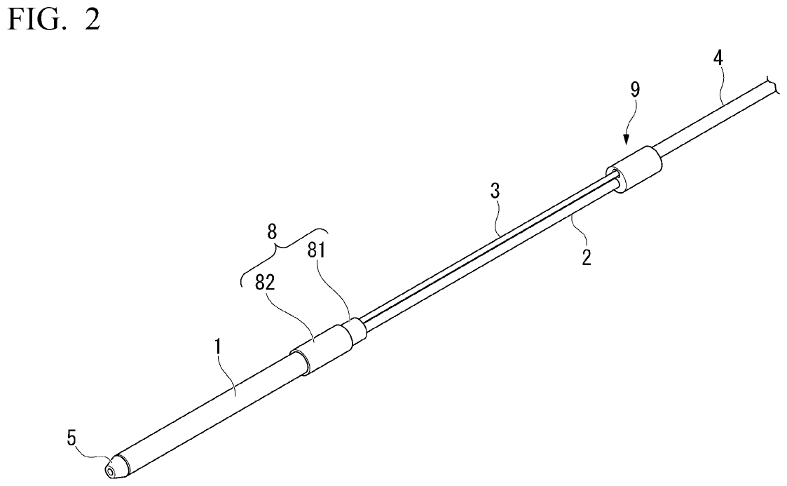

The delivery device 100 will be described with reference to FIGS. 2 to 11. As shown in FIG. 2, the delivery device 100 has an elongated shape as a whole. The delivery device 100 includes a first outer tube member 1, an inner tube member 2, a pulling member 3, a second outer tube member 4, a tip 5, a stent 6, a first joint portion 8, and a second joint portion 9.

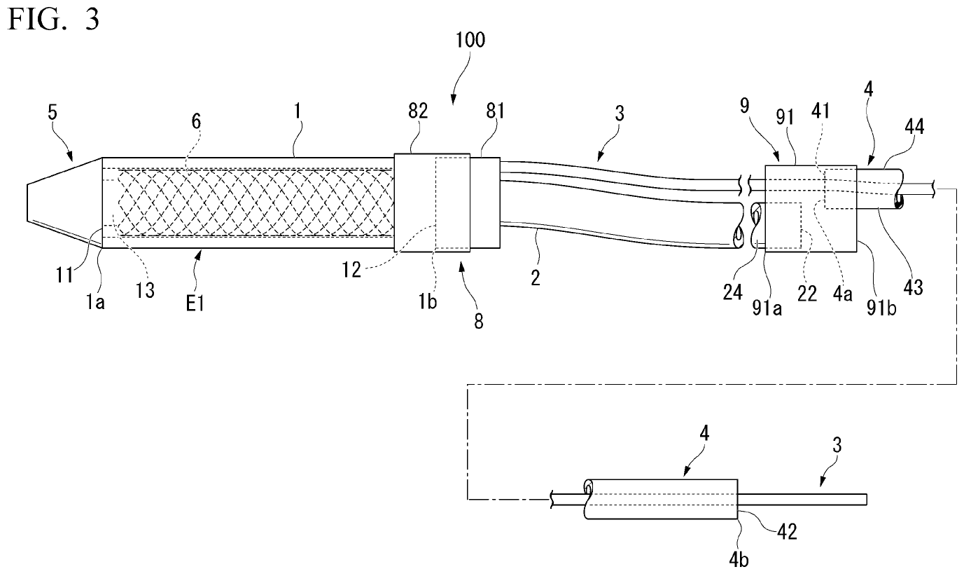

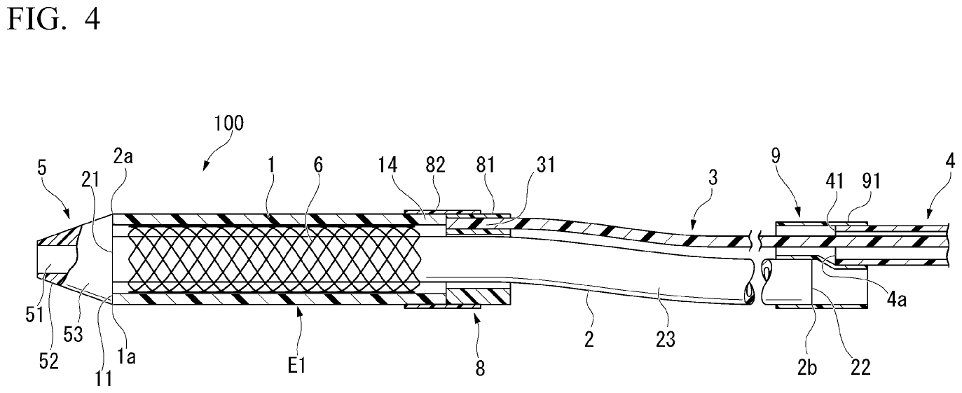

The first outer tube member 1 is a long tubular member that is capable of being inserted into the treatment instrument channel 230 of the endoscope 200. The first outer tube member 1 is formed of a resin or the like and has flexibility. The first outer tube member 1 may be a tube formed of a resin or the like, or may be a coil sheath. As shown in FIGS. 3 and 4, the first outer tube member 1 has a first end 1a on the distal side of the delivery device 100 and a second end 1b on the proximal side of the delivery device 100. A first opening 11 and a second opening 12 are formed in the first outer tube member 1. The first opening 11 opens at the first end 1a. The second opening 12 opens at the second end 1b. The first opening 11 and the second opening 12 communicate with an internal space (a lumen) 13 of the first outer tube member 1. The first opening 11 and the second opening 12 are substantially circular openings through which the inner tube member 2 is capable of being inserted.

The inner tube member 2 is a long tubular member that can be inserted into the treatment instrument channel 230 of the endoscope 200. The inner tube member 2 is formed of a resin or the like and has flexibility. The inner tube member 2 may be a tube formed of a resin or the like, or may be a coil sheath. As shown in FIGS. 3 and 4, the inner tube member 2 has a first end 2a on the distal side of the delivery device 100 and a second end 2b on the proximal side of the delivery device 100. A first opening 21 and a second opening 22 are formed in the inner tube member 2. The first opening 21 opens at the first end 2a. The second opening 22 opens at the second end 2b. The first opening 21 and the second opening 22 communicate with a lumen (a guide wire lumen) 23 of the inner tube member 2. The first opening 21 and the second opening 22 are substantially circular openings through which a guide wire G is capable of being inserted. The inner tube member 2 is inserted into the first outer tube member 1. Specifically, the inner tube member 2 passes through the first opening 11 and the second opening 12 and is inserted into the lumen 13 of the first outer tube member 1 to be relatively movable. The outer diameter of the inner tube member 2 is smaller than the inner diameter of the lumen 13 of the first outer tube member 1.

The first outer tube member 1 has the stent storage region E1 in which the stent 6 is capable of being stored. The stent storage region E1 is a region for storing the stent 6 between the first outer tube member 1 and the inner tube member 2. The stent storage region E1 is a region for storing the stent 6, into which the inner tube member 2 is inserted, in the lumen 13 of the first outer tube member 1. The stent storage region E1 is a region that extends from the first end 1a of the first outer tube member 1 toward the proximal side by the length of the stent 6 or longer.

The second outer tube member 4 is a long tubular member that is capable of being inserted into the treatment instrument channel 230 of the endoscope 200. The second outer tube member 4 is formed of a resin or the like and has flexibility. The second outer tube member 4 may be a tube formed of a resin or the like, or may be a coil sheath. As shown in FIGS. 3 and 4, the second outer tube member 4 has a first end 4a on the distal side of the delivery device 100, and has a second end 4b on the proximal side of the delivery device 100. The second outer tube member 4 is provided on the proximal side of the delivery device 100 with respect to the first outer tube member 1. A first opening 41 and a second opening 42 are formed in the second outer tube member 4. The first opening 41 opens at the first end 4a. The second opening 42 opens at the second end 4b. The first opening 41 and the second opening 42 communicate with an internal space (a lumen) 43 of the second outer tube member 4. The first opening 41 and the second opening 42 are substantially circular openings through which the pulling member 3 is capable of being inserted. The first end 4a of the second outer tube member 4 is connected to the second end 2b of the inner tube member 2 by the second joint portion 9.

The pulling member 3 is a long member that is capable of being inserted into the treatment instrument channel 230 of the endoscope 200. The pulling member 3 is a wire that is formed of a metal such as NiTi or a resin and has flexibility. As shown in FIGS. 3 and 4, the pulling member 3 is inserted into the second outer tube member 4. Specifically, the pulling member 3 passes through the first opening 41 and the second opening 42 and is inserted into the internal space (the lumen) 43 of the second outer tube member 4 to be relatively movable. The outer diameter of the pulling member 3 inserted into the internal space (the lumen) 43 of the second outer tube member 4 is smaller than the inner diameter of the lumen 43 of the second outer tube member 4. A distal end portion 31 of the pulling member 3 is connected to a proximal end portion 14 of the first outer tube member 1 by the first joint portion 8. The surface of at least the distal end portion 31 of the pulling member 3 may be roughened by a technique such as sandblasting. An operator can pull the first outer tube member 1 toward the proximal side by pulling the pulling member 3.

The tip 5 has a substantially conical shape. A through hole 51 extending in an axial direction is formed in the tip 5. As shown in FIGS. 3 and 4, the tip 5 has a distal end portion 52 and a proximal end portion 53. The tip 5 is connected to the inner tube member 2 at the proximal end portion 53. The distal end portion 52 has a smaller diameter than the proximal end portion 53. The through hole 51 communicates with the guide wire lumen 23 of the inner tube member 2 via the first opening 21. For this reason, when the guide wire G is inserted into the through hole 51 of the tip 5, the guide wire G can enter the guide wire lumen 23 of the inner tube member 2.

The guide wire is an instrument used when the delivery device 100 is inserted into a lumen. For example, the operator inserts the proximal end portion of the guide wire G protruding from the forceps plug 225 of the endoscope 200 into the through hole 51 of the tip 5 of the delivery device 100. The guide wire G enters the guide wire lumen 23 of the inner tube member 2 through the through hole 51. The operator pushes the delivery device 100 while holding the guidewire G, thereby the delivery device 100 advances along the guide wire G.

The stent 6 is a tubular self-expanding stent. The stent 6 is formed by weaving a wire. As shown in FIGS. 3 and 4, the stent 6 is accommodated in the stent storage region E1 of the first outer tube member 1. Specifically, the inner tube member 2 is passed through the interior of the stent 6, and the stent 6 in a reduced diameter state is accommodated in a gap between the inner tube member 2 and the first outer tube member 1. The stent 6 is locked to a locking portion (not shown) formed on the outer circumferential surface of the inner tube member 2. As a result, in the reduces diameter state, the stent 6 is positioned with respect to the inner tube member 2 and does not move relative to the inner tube member 2 in a longitudinal direction. By the pulling member 3 being pulled toward the proximal side, the first outer tube member 1 moves toward the proximal side of the delivery device 100 and the stent 6 is configured to be released from the delivery device 100. The stent 6 may be a laser-cut type stent formed by cutting a metal tube with a laser. The stent 6 may be a covered stent in which the inner circumferential surface or the outer circumferential surface of the inner tube member 2 is covered with a resin film.

The wire for forming the stent 6 is a superelastic alloy containing NiTi as a main material. A superelastic alloy containing NiTi as a main material is not permanently deformed when it is woven, and the woven shape is memorized by applying a heat treatment in a woven state.

Although an example in which the stent 6 is a self-expanding stent has been described, the stent 6 is not limited to the self-expanding stent. The stent 6 may be a non-self-expanding stent, and examples of the stent 6 include a CoCr-based alloy stent, a biodegradable stent made of polylactic acid, polyglycolic acid, and their copolymers, and the like. The stent 6 may be a fluid-expanding stent. Examples of the fluid-expanding stent include a non-self-expanding stent that is expanded by other treatment instruments such as a balloon.

The first joint portion 8 has a first joint member 81 and a fixing member 82. The first joint portion 8 connects and fixes the first outer tube member 1 and the pulling member 3 to each other.

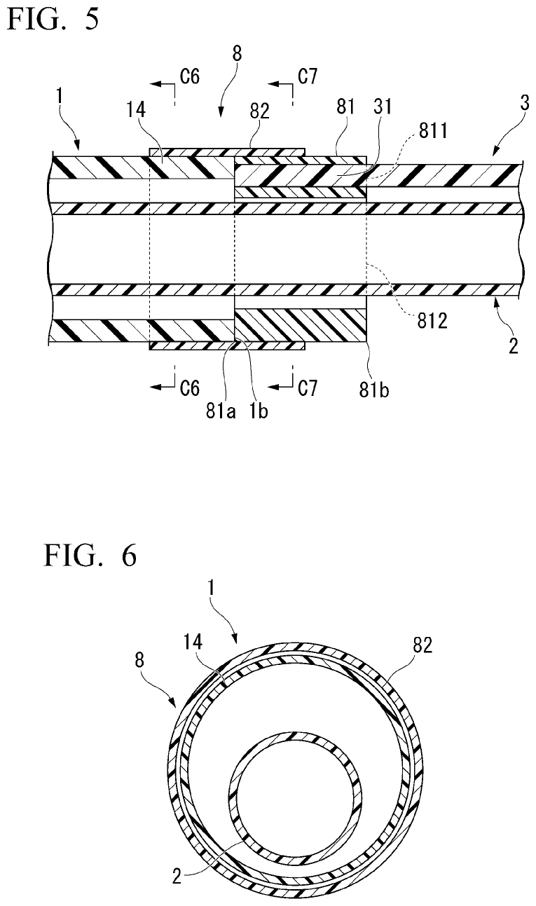

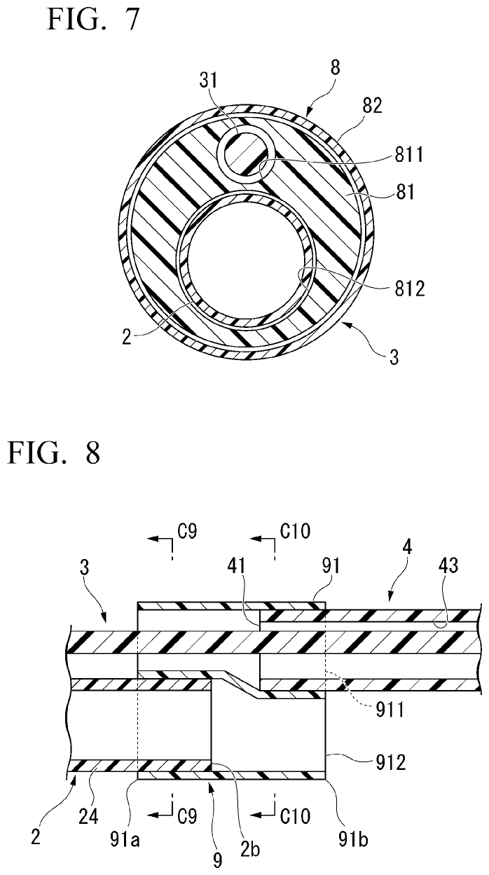

The first joint member 81 is a tubular member that is capable of being inserted into the treatment instrument channel 230 of the endoscope 200. The first joint member 81 is formed of a resin or the like and has flexibility. The entire first joint member 81 or the surface of the first joint member 81 is formed of the same resin as the surface of the first outer tube member 1. However, the material of the entire first joint member 81 or the surface of the first joint member 81 is not limited to this. As shown in FIG. 5, the first joint member 81 has a first end 81a on the distal side of the delivery device 100 and a second end 81b on the proximal side of the delivery device 100. As shown in FIG. 7, the first joint member 81 is a multi-lumen tube having a first hole 811 and a second hole 812 formed therein. The first end 81a of the first joint member 81 is in contact with the second end 1b of the first outer tube member 1. The end surface of the first end 81a of the first joint member 81 is in contact with the end surface of the second end 1b of the first outer tube member 1.

The distal end portion 31 of the pulling member 3 is inserted into the first hole 811, and the distal end portion 31 is fixed in the first hole 811. Specifically, the distal end portion 31 is inserted into the first hole 811 and then is fixed to the first hole 811 with an adhesive. At this time, it is possible to improve the joining strength by roughening the surface of the pulling member 3. That is, when the surface roughness of the pulling member 3 is large, the joining strength is improved. The first hole 811 and the pulling member 3 may be fixed to each other with a frictional force generated between the inner surface of the first hole 811 and the outer surface of the pulling member 3. The inner diameter of the first hole 811 is approximately equal to the outer diameter of the pulling member 3.

As shown in FIGS. 5 and 7, the inner tube member 2 is inserted into the second hole 812. The inner tube member 2 is inserted into the second hole 812 to be relatively movable. The inner diameter of the second hole 812 is larger than the outer diameter of the inner tube member 2. In the present embodiment, the inner diameter of the second hole 812 is larger than the inner diameter of the first hole 811, but the present invention is not limited to this. That is, the inner diameter of the second hole 812 may be smaller than the inner diameter of the first hole 811.

The fixing member 82 is a tubular member that is capable of being inserted into the treatment instrument channel 230 of the endoscope 200. The fixing member 82 is formed of a resin or the like and has flexibility. The fixing member 82 is provided to cover the first outer tube member 1 and the first joint member 81. Specifically, the fixing member 82 is provided to be disposed at a radial direction outer side of the first outer tube member 1 and the first joint member 81, to cover the entire circumference of the first outer tube member 1 and the entire circumference of the first joint member 81, and to bring the first outer tube member 1 and the first joint member 81 into close contact with each other. The fixing member 82 covers the entire circumferences of the second end 1b of the first outer tube member 1 and the first end 81a of the first joint member 81. The fixing member 82 fixes the first outer tube member 1 and the first joint member 81 to each other.

It is desirable that the entire fixing member 82 or the surface of the fixing member 82 be formed of the same material as the surface of the first outer tube member 1. It is desirable that the entire fixing member 82 or the surface of the fixing member 82 be formed of the same resin as the surface of the first joint member 81. That is, it is desirable that the surface of the first joint member 81, the surface of the fixing member 82, and the surface of the first outer tube member 1 are formed of the same material, especially the same resin. In the present invention, this resin may have a heat-fusing property. The surface of the first joint member 81, the surface of the fixing member 82, and the surface of the first outer tube member 1 are easily heat-fused by being formed of a resin having a heat-fusing property. For this reason, joining is stronger compared to joining based on the elasticity of each member. However, the material of the fixing member 82 or the surface of the fixing member 82 is not limited to this.

A structure in which the first outer tube member 1 and the pulling member 3 are connected and fixed to each other by the first joint portion 8 will be described. The distal end portion 31 of the pulling member 3 is fixed in the first hole 811 of the first joint member 81. The end surface of the first end 81a of the first joint member 81 and the end surface of the second end 1b of the first outer tube member are butted against each other. That is, the end surface of the first end 81a of the first joint member 81 and the end surface of the second end 1b of the first outer tube member are in contact with each other. The fixing member 82 is provided to cover both of the first outer tube member 1 and the first joint member 81. The fixing member 82 covers the entire circumferences of the second end 1b of the first outer tube member 1 and the first end 81a of the first joint member 81 to bring the first outer tube member 1 and the first joint member 81 into close contact with each other.

With the structure described above, the first joint portion 8 connects and fixes the first outer tube member 1 and the pulling member 3 to each other. For this reason, compared to the case where the first outer tube member 1 and the pulling member 3 are connected and fixed to each other at only one point, a joining area can be secured. The first joint portion 8 has a structure in which the fixing member 82 covers the entire circumferences of the first outer tube member 1 and the first joint member 81. For this reason, compared to the case where a part of the outer circumferential surface of the tubular member such as the first outer tube member 1 is joined to a part of the inner circumferential surface of the fixing member 82, a joining area is capable of being secured.

The second joint portion 9 has a second joint member 91. The second joint portion 9 connects and fixes the inner tube member 2 and the second outer tube member 4 to each other.

The second joint member 91 is a tubular member that is capable of being inserted into the treatment instrument channel 230 of the endoscope 200. The second joint member 91 is formed of a resin or the like and has flexibility. As shown in FIGS. 8 to 10, the second joint member 91 has a first end 91a on the distal side of the delivery device 100 and has a second end 91b on the proximal side of the delivery device 100. The second joint member 91 connects and fixes the inner tube member 2 and the second outer tube member 4 to each other. The second joint member 91 is a multi-lumen tube having a first hole 911 and a second hole 912.

The first end 4a of the second outer tube member 4 is inserted into the first hole 911, and the first end 4a of the second outer tube member 4 is fixed in the first hole 911. The pulling member 3 is inserted into the first hole 911. The pulling member 3 is inserted into the first hole 911 to be relatively movable. As shown in FIGS. 8 to 10, the inner diameter of the first hole 911 differs between the first end 91a and the second end 91b of the second joint member 91. Specifically, in the first hole 911, the inner diameter of the second end 91b is larger than the inner diameter of the first end 91a. The inner diameter of the first hole 911 at the second end 91b is approximately equal to the outer diameter of the second outer tube member 4.

The second end 2b of the inner tube member 2 is inserted into the second hole 912, and the second end 2b of the inner tube member 2 is fixed in the second hole 912. The second hole 912 is configured such that the guide wire G is capable of being inserted thereinto. The guide wire G is capable of being inserted into the second hole 912 so as to be relatively movable. The inner diameter of the second hole 912 of the first end 91a of the second joint member 91 is different from the inner diameter of the second end 91b of the second joint member 91. Specifically, in the second hole 912, the inner diameter of the second end 91b is smaller than the inner diameter of the first end 91a. The inner diameter of the second hole 912 at the first end 91a is approximately equal to the outer diameter of the inner tube member 2.

A structure in which the inner tube member 2 and the second outer tube member 4 are connected and fixed to each other by the second joint portion 9 will be described. The first end 4a of the second outer tube member 4 is fixed in the first hole 911 of the second joint member 91. The second end 2b of the inner tube member 2 is fixed in the second hole 912 of the second joint member 91.

With the structure described above, the second joint portion 9 connects and fixes the inner tube member 2 and the second outer tube member 4 to each other. For this reason, compared to the case where the inner tube member and the second joint member are connected and fixed to each other at only one point, a wider joining area is capable of being secured. Furthermore, the second joint portion 9 has a structure in which the first hole 911 of the second joint member 91 covers the entire circumference of the second outer tube member 4 and the second hole 912 of the second joint member 91 covers the entire circumference of the inner tube member 2. For this reason, compared to the case where a part of the outer circumferential surface of the inner tube member such as the second outer tube member 4 is joined to a part of the inner circumferential surface of the tubular member, a wider joining area is capable of being secured.

As described above, the inner diameter of the first hole 911 and the inner diameter of the second hole 912 of the second joint member 91 differ at each of the first end 91a and the second end 91b of the second joint member 91, respectively. The inner diameter of the first hole 911 at the second end 91b is a diameter that allows the second outer tube member 4 to be inserted into the first hole 911. The inner diameter of the first hole 911 at the first end 91a is small enough to allow the pulling member 3 having an outer diameter smaller than that of the second outer tube member 4 to be inserted into the first hole 911. Similarly, the inner diameter of the second hole 912 at the first end 91a is a diameter that allows the inner tube member 2 to be inserted into the second hole 912, and the inner diameter of the second hole 912 at the second end 91b is small enough to allow the guide wire G having an outer diameter smaller than that of the inner tube member 2 to be inserted into the second hole 912. For this reason, compared to the case where the diameter of the first hole 911 and the diameter of the second hole 912 are uniform between the first end 91a and the second end 91b, the outer diameter of the second joint member 91 is capable of being made smaller.

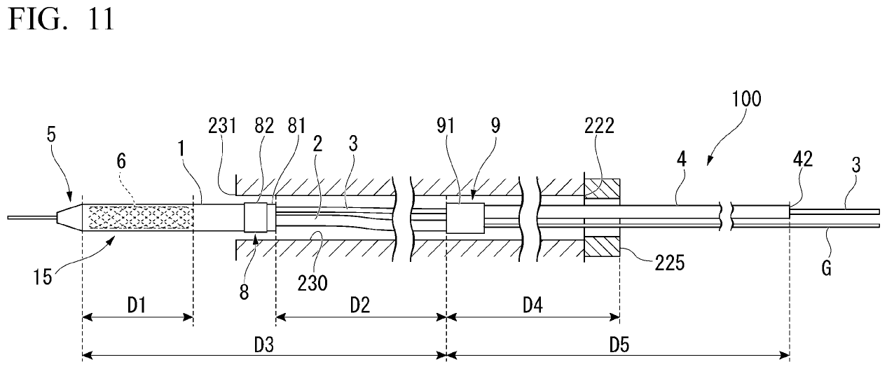

A usage action of the delivery device 100 described above will be described with reference to FIG. 11. The distal end of the delivery device 100 protrudes from the distal end portion 231 of the treatment instrument channel 230 of the endoscope 200 during use. In particular, in a state in which the distal end of the delivery device 100 is disposed near the placement position of the stent 6, the entire stent storage region E1 provided in the delivery device 100 protrudes from the distal end portion 231 of the treatment instrument channel 230. In this state, the first joint member 81 and the fixing member 82 are disposed in the treatment instrument channel 230. Similarly, the second joint member 91 is also disposed in the treatment instrument channel 230. On the other hand, the second opening 42 of the second outer tube member 4 is disposed outside from the forceps port 222 on the proximal side of the treatment instrument channel 230.

After determining the indwell position of the stent 6, the operator and his/her assistant then pull the pulling member 3 toward the proximal side. The pulling member 3 is pulled toward the proximal side in a state in which the position of the second outer tube member 4 with respect to the forceps plug 225 is fixed. The pulling member 3 is fixed to the first outer tube member 1 by the first joint member 81 and the fixing member 82. The inner tube member 2 is inserted into the lumen 13 so as to be advanceable and reatractable with respect to the first outer tube member 1. For this reason, when the pulling member 3 is pulled toward the proximal side, the first outer tube member 1, the first joint member 81, and the fixing member 82 retract in accordance with the movement of the pulling member 3, and the inner tube member 2 does not retract. The inner tube member 2 is connected to the second outer tube member 4, and the second outer tube member 4 is held in position with respect to the treatment instrument channel 230. For this reason, the position of the inner tube member 2 is held, and the position of the stent storage region E1 is less likely to deviate from the target position where it is disposed. In this state, when the first outer tube member 1 retracts with respect to the inner tube member 2, the stent 6 is exposed to the outside. When the stent 6 is exposed to the outside from the stent storage region E1, the diameter of the stent 6 is increased to be larger than the outer diameter of the tip 5. Thereafter, when the pulling member 3 is further pulled toward the proximal side, the first joint portion 8 comes into contact with the second joint portion 9. When the pulling member 3 is continued to be pulled in a state in which the first joint portion 8 is in contact with the second joint portion 9, the inner tube member 2 and the tip 5 retract. Since the diameter of the stent 6 is increased to be larger than the outer diameter of the tip 5, the delivery device 100 is capable of being retracted without the stent 6 being retracted.

Until the stent 6 is completely expanded, it is possible to re-accommodate (re-capture) the stent 6 between the first outer tube member 1 and the inner tube member 2 by advancing the first outer tube member 1 with respect to the inner tube member 2 while reducing the diameter of the stent 6. Recapture is useful when resetting the placement position.

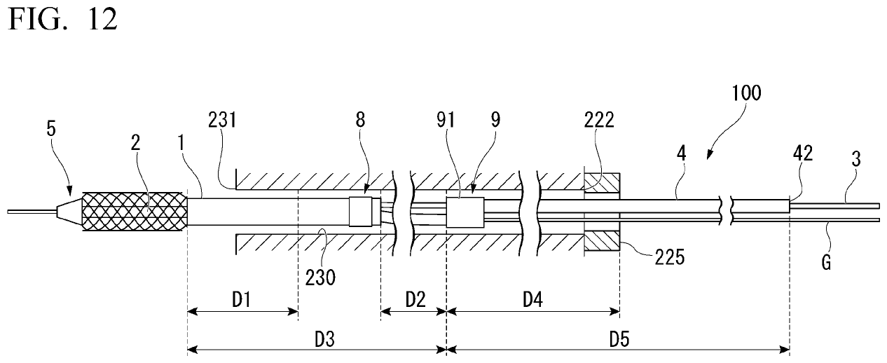

The dimensions of the members constituting the delivery device 100 in the longitudinal direction of the delivery device 100 will be described with reference to FIGS. 11 and 12.

The length of the stent storage region E1 in the longitudinal direction is referred to as a length D1. The length from the second end 81b of the first joint member 81 to the first end 91a of the second joint member 91 in an initial state in which the stent 6 is stored in the stent storage region E1 and the stent 6 can be indwelled is referred to as a length D2. The length D2 is longer than the length D1. The length D2 is a length when the first outer tube member 1 is farthest away from the second joint portion 9 toward the distal side. The length D2 is a length of a portion of the inner tube member 2 exposed in the treatment instrument channel 230. With the above structure, even in a case where the pulling member 3 is pulled toward the proximal side until the stent 6 is released from the delivery device 100, the first joint member 81 and the second joint member 91 do not come into contact with each other. Therefore, it is prevented from being in a situation that the first outer tube member 1 is not sufficiently retracted by which the first joint member 81 cannot be pulled toward the proximal side of the delivery device 100, and the stent 6 is not completely exposed from the stent storage region E1, and thus the stent 6 cannot be released. In a region of the length D2, the members constituting the delivery device 100, i.e. the inner tube member 2 and the pulling member 3, are less likely to come into contact with the inside of the treatment instrument channel 230. For this reason, when the delivery device 100 advances and retracts with respect to the treatment instrument channel 230, it is less likely to be affected by the frictional force on the inner surface of the treatment instrument channel 230.

The second joint member 91 is inserted into the treatment instrument channel 230. Therefore, the second joint member 91 is disposed on the distal side with respect to the forceps port 222. In the initial state, that is, in a state in which the first end 1a and the stent storage region E1 of the first outer tube member 1 protrude from the distal end of the treatment instrument channel 230 and the second end 1b is located within the distal end portion 231, a length D4 from the first end 91a of the second joint member 91 to the forceps port 222 of the endoscope 200 is longer than a length D3 from the first end 1a of the first outer tube member 1 to the first end 91a of the second joint member 91. Similarly, a length D5 from the first end 91a of the second joint member 91 to the second end 4b of the second outer tube member 4 is longer than the length D3. The length D4 is an insertion length of the second outer tube member 4 inserted into the treatment instrument channel 230.

When the stent 6 is indwelled, the position of the second outer tube member 4 with respect to the treatment instrument channel 230 is held, and the length D2 and the length D3 change. Here, a member that moves when the delivery device 100 releases the stent 6 is referred to as a moving member. Specifically, examples of the moving member include the first joint member 81, the first joint portion 8, and the pulling member 3. The length D2 and the length D3 vary depending on the degree of movement of the moving member. The length D3 is a length such that the first end 81a of the first joint portion 8 is not exposed from the distal end portion 231 of the treatment instrument channel 230 even in a case where the pulling member 3 is pushed out to the most distal side. For example, in the case of delivery device 100 for the bile duct, the length D3 is 20 cm. The length D3 is longer than the total length of the length of the first outer tube member 1 and the maximum value of the length D2.

As mentioned above, the length D4 is longer than the length D3. That is, in the delivery device 100, the length D3 of a portion that advances and retracts within the long treatment instrument channel 230 is shorter than the length D4 of a portion where the position is held. As a result, it becomes possible to reduce the influence of friction generated between the delivery device 100 and the treatment instrument channel 230 during the placement operation of the stent 6. For this reason, the position of the delivery device 100 is less likely to deviate, and the stent 6 is capable of being indwelled at a desired position.

As mentioned above, the length D5 is longer than the length D3. That is, in the delivery device 100, a length when the length D3 of a portion that advances and retracts within the long treatment instrument channel 230 is the maximum length (a maximum value of the length D3) is shorter than the length D5. As a result, it becomes possible to reduce the influence of friction generated between the delivery device 100 and the treatment instrument channel 230 during the placement operation of the stent 6. For this reason, the position of the delivery device 100 is less likely to deviate with respect to the treatment instrument channel 230, and the stent 6 is capable of being indwelled at a desired position.

Since the lengths D3, D4, and D5 have the relationship as described above, regions where the moving member is not exposed to the outside from the distal end portion of the delivery device 100 (a region of D4 and D5) is larger than a region where the moving member is exposed to the outside from the distal end portion of the delivery device 100 (a region of 13). For this reason, when the delivery device 100 releases the stent 6, it is possible to curb the influence of a frictional force generated between the delivery device 100 and the treatment instrument channel 230.

According to the delivery device 100 related to the embodiment, the pulling member 3 is fixed in the first hole 911. The first joint member 81 in which the first hole 811 is formed and the first outer tube member 1 are fixed by the fixing member 82. That is, the pulling member 3 is fixed to the first outer tube member 1 by the first joint member 81 and the fixing member 82. By fixing the pulling member 3 in the first hole 911, a wide joining area between the pulling member 3 and the first hole 911 is capable of being secured. As a result, compared to the case where the end surface of the pulling member and the end surface of the joint member are fixed at only one point, a wider joining area is capable of being secured and the joining strength is capable of being increased.

According to the delivery device 100 related to the embodiment, the first outer tube member 1, the first joint member 81, and the fixing member 82 are fixed to each other. That is, the fixing member 82 covers and close contact with the entire circumferences of the first outer tube member 1 and the first joint member 81. Therefore, a wider joining area is capable of being secured.

According to the delivery device 100 related to the embodiment, the first outer tube member 1 and the first joint member 81 are connected to each other at their end surfaces on the radial direction inner side of the fixing member 82. As a result, the inner diameter of the delivery device 100 is capable of being secured, and the inner diameter of the second hole 812 into which the inner tube member 2 is inserted so as to be capable of advancing and retracting is capable of being secured widely. As a result, when the pulling member 3 is pulled, the first outer tube member 1 is smoothly pulled with respect to the inner tube member 2. In addition, as a result of securing a wide inner diameter of the second hole 812, it is possible to secure a wide advancing and retracting path for the guide wire. Therefore, the guide wire G is smoothly advanced and retracted.

According to the delivery device 100 related to the embodiment, since the fixing member 82, the first outer tube member 1, and the first joint member 81 are formed of the same material, they is capable of being integrated and joined with each other by heat fusing and is capable of being easily joined with each other.

According to the delivery device 100 related to the embodiment, the diameter of the first hole 911 has a size that allows the pulling member to be fixed therein, and the diameter of the second hole 912 has a size that allows the inner tube member 2 to be inserted into the second hole 912 so as to be capable of advancing and retracting. Therefore, the inner tube member 2 is easily advanced and retracted while the pulling member is fixed at the first hole 911.

According to the delivery device 100 related to the embodiment, the second joint member 91 is a member that connects the inner tube member 2 and the second outer tube member 4 to each other. As a result, compared to the case where the inner tube member 2 and the second outer tube member 4 are joined at only one point, a wider joining area is capable of being secured.

According to the delivery device 100 related to the embodiment, the stent 6 is released from the stent storage region E1 by pulling the pulling member. The first hole 911 of the second joint member 91 has a size that allows the pulling member to advance and retract. Therefore, it is possible to secure a path for the pulling member so as to be capable of advancing and retracting while the inner tube member 2 and the second outer tube member 4 are joined. As a result, the path for moving the pulling member advancing and retracting is secured while the outer diameter dimension of the delivery device 100 is suppressed, and the pulling member is smoothly advanced and retracted, resulting in excellent operability.

According to the delivery device 100 related to the embodiment, the entire delivery device 100 advances and retracts along the guide wire. The guide wire is provided so as to be capable of advancing and retracting with respect to the inner tube member 2. Therefore, a wide advancing and retracting path for the guide wire G is capable of being ensured while the inner tube member 2 and the second outer tube member 4 are reliably joined with each other over a wide joining area.

According to the delivery device 100 related to the embodiment, the inner diameter of the first hole 911 at the first end 91a only has to be a diameter that allows the pulling member 3 to be inserted into the first hole 911, but the inner diameter of the first hole 911 at the second end 91b has to be a diameter that allows the second outer tube member 4 to be inserted into and fixed to the first hole 911. That is, the diameter of the first hole 911 at the second end 91b is larger than the diameter of the first hole 911 at the first end 91a. The inner diameter of the second hole 912 at the first end 91a has to be a diameter that allows the inner tube member 2 to be inserted into and fixed to the second hole 912, but the inner diameter of the second hole 912 at the second end 91b only has to be a diameter that allows the guide wire G to be inserted into the second hole 912. As a result, the outer diameter of the second joint member 91 is capable of being reduced. As a result of being able to reduce the diameter of the second joint portion 9, the second joint portion 9 is less likely to come into contact with the treatment instrument channel 230 when the delivery device 100 advances and retracts within the treatment instrument channel 230, and thus the advance and retraction of the delivery device 100 is smoothly performed. In particular, it is possible to prevent the treatment instrument channel 230 and the second joint portion 9 from coming into contact with each other when the insertion section of the endoscope is bent and providing resistance to the advancing and retracting movement.

The first outer tube member 1, the inner tube member 2, the pulling member 3, the second outer tube member 4, the first joint member 81, the fixing member 82, and the second joint member 91 are not particularly limited in material as long as the desired mechanical properties are satisfied. In a case of delivery devices to be used under fluoroscopy, radiopaque metallic markers (for example, medical radiopaque metals and alloys such as platinum, tungsten, and iridium) may be added to the above members or radiopaque materials (for example, barium sulfate and the like) may be mixed into the above members.

In the above, one embodiment has been described in detail with reference to the drawings, but the specific configuration is not limited to the embodiment, and a design change and the like within a range not departing from the gist of the present invention are also included. In addition, the constituent elements shown in the above-described embodiment and a modification example shown below can be appropriately combined and configured.

What is claimed is:

1. A delivery device comprising:
a first outer tube member;
an inner tube member that is inserted into the first outer tube member;
a second outer tube member that is positioned at a proximal side with respect to the first outer tube member and is connected to the inner tube member;
a pulling member that is inserted into the second outer tube member and is connected to the first outer tube member;
a first joint member in which a first hole for fixing the pulling member therein and a second hole configured to allow the inner tube member to advance and retract are formed; and
a fixing member that fixes the first joint member to the first outer tube member to each other.

2. The delivery device according to claim 1, wherein the fixing member is positioned at a radial direction outer side of the first outer tube member and the first joint member, and covers the entire circumference of an outer circumferential surface of the first outer tube member and the entire circumference of an outer circumferential surface of the first joint member to fix the first outer tube member and the first joint member to each other.

3. The delivery device according to claim 2, wherein an end surface of the first outer tube member and an end surface of the first joint member are disposed to come into contact with each other on the radial direction inner side of the fixing member.

4. The delivery device according to claim 2,
wherein a surface of the fixing member, a surface of the first outer tube member, and a surface of the first joint member are formed of the same resin having a heat-fusing property, and
wherein the fixing member, the first outer tube member, and the first joint member are fixed to each other by heat fusing.

5. The delivery device according to claim 1, wherein a diameter of the first hole of the first joint member is smaller than a diameter of the second hole of the first joint member.

6. The delivery device according to claim 1, further comprising a second joint member in which a first hole configured to fix the second outer tube member therein and a second hole configured to fix the inner tube member therein are formed.

7. The delivery device according to claim 6, wherein the first hole of the second joint member is configured to allow the pulling member to be inserted thereinto and allows the pulling member to advance and retract.

8. The delivery device according to claim 6, wherein the second hole of the second joint member has a size that allows a guide wire for guiding the delivery device to be inserted thereinto and allows the guide wire to advance and retract.

9. The delivery device according to claim 6,
wherein the second joint member has a first end in which the inner tube member is fixed therein and a second end in which the second outer tube member is fixed therein,
wherein a diameter of the first hole is larger at the second end than at the first end, and
wherein a diameter of the second hole is larger at the first end than at the second end.

10. The delivery device according to claim 1, further comprising a second joint member that is disposed at a proximal side of the delivery device with respect to the first joint member and fixes the second outer tube member with the inner tube member.

11. The delivery device according to claim 10,
wherein the first outer tube member has a stent storage region in which the stent is stored,
wherein the first joint member has a second end at a proximal side of the delivery device,
wherein the second joint member has a first end at a distal side of the delivery device, and
wherein, in a longitudinal direction of the delivery device, a length from the second end of the first joint member to the first end of the second joint member is longer than a length of the stent storage region.

12. The delivery device according to claim 10,
wherein the first outer tube member has a first end at a distal side of the delivery device,
wherein the second joint member has a first end at a distal side of the delivery device,
wherein the second outer tube member has a second end at a proximal side of the delivery device, and
wherein, in a longitudinal direction of the delivery device, a length from the first end of the second joint member to the second end of the second outer tube member is longer than a length from the first end of the first outer tube member to the first end of the second joint member.

13. The delivery device according to claim 1, further comprising a stent that is stored between the first outer tube member and the inner tube member and is released as the pulling member moves toward a proximal side with respect to the second outer tube member.

14. An endoscope system comprising:
a delivery device including
a first outer tube member,
an inner tube member that is inserted into the first outer tube member,
a second outer tube member that is positioned at a proximal side with respect to the first outer tube member and is connected to the inner tube member,
a pulling member that is inserted into the second outer tube member and is connected to the first outer tube member,
a first joint member that has a first hole for fixing the pulling member therein and a second hole configured to allow the inner tube member to be inserted thereinto and to allow the inner tube member to advance and retract, and
a fixing member that fixes the first joint member to the first outer tube member; and
an endoscope having a treatment instrument channel configured to allow the delivery device to advance and retract.

15. The endoscope system according to claim 14, wherein the delivery device further includes a second joint member that is disposed at a proximal side of the delivery device with respect to the first joint member and fixes the second outer tube member with the inner tube member.

16. The endoscope system according to claim 15, wherein the delivery device further includes a stent that is stored between the first outer tube member and the inner tube member, and is released as the pulling member moves toward a proximal side with respect to the second outer tube member.

17. The endoscope system according to claim 16,
wherein the first joint member has a stent storage region in which the stent is stored, and
wherein the first joint member and the second joint member are configured to be positioned in the treatment instrument channel in a state in which the entire stent storage region protrudes from a distal side of the endoscope in the treatment instrument channel.

18. The endoscope system according to claim 16,
wherein the first outer tube member has a first end at a distal side of the delivery device,
wherein the second joint member has a first end at a distal side of the delivery device,
wherein the first joint member has a stent storage region in which the stent is stored, and
wherein, in a longitudinal direction of the delivery device, a length from the first end of the second joint member to a forceps port of the endoscope is longer than a length from the first end of the first outer tube member to the first end of the second joint member in a state in which the entire stent storage region protrudes from a distal side of the endoscope in the treatment instrument channel.
